(19) United States
(12) Patent Application Publication
(10) Pub. No.: US 2010/0185254 A1
(43) Pub. Date: Jul. 22, 2010

(54) ELECTRONIC CONTINUOUS OR PERIODIC AIRWAY THERAPY (ECAT) FOR SLEEP -BREATHING DISORDERS

(75) Inventors: Sherrill F. Lindquist, Melbourne, FL (US); John E. Zurasky, Merritt Island, FL (US); Jacob D. Zurasky, Merritt Island, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: ZURLIN TECHNOLOGIES HOLDINGS, LLC, Melbourne, FL (US)

(21) Appl. No.: 12/690,515

(22) Filed: Jan. 20, 2010

Related U.S. Application Data
(63) Continuation-in-part of application No. 12/154,339, filed on May 22, 2008.
(60) Provisional application No. 60/946,159, filed on Jun. 26, 2007.
(60) Provisional application No. 61/146,087, filed on Jan. 21, 2009.

(51) Int. Cl. A61N 1/36

(52) U.S. Cl. 607/42

(57) ABSTRACT

The intra-oral electronic therapy device includes a substrate to be positioned in a patient's mouth, a rechargeable battery carried by the substrate, and at least one hamular notch tissue contact electrode extending outwardly from the substrate to contact at least one hamular notch in the patient's mouth. A controller is carried by the substrate and cooperates with the rechargeable battery and the hamular notch tissue contact electrode to provide a predetermined electrical stimulation pattern to a hamular notch in the patient's mouth. A programming interface is carried by the substrate and coupled to the controller to permit programming of the predetermined stimulation pattern therein.

### CROSS-REFERENCE TO RELATED APPLICATIONS

The present application is a Continuation-in-Part (CIP) of U.S. patent application Ser. No. 12/154,339 filed May 22, 2008 (now U.S. Patent Application Publication 2009/0082839, and which also claims priority to U.S. Provisional Application No. 60/946,159 filed Jun. 26, 2007 entitled “Electronic Anti-Snoring & Sleep Apnea Device (EAS/SAD) For Sleep-Breathing Disorders, Electronic Anti-Bruxing Device, And Electronic Device For TMD Therapy”), and claims priority from U.S. Provisional Application No. 61/146,087, filed Jan. 21, 2009, entitled “Electronic Continuous or Periodic Therapy Device (ECAT) For Sleep-Breathing Disorders, Bruxing disorders, And TMJ Disorders” by Lindquist et al., which is hereby incorporated by reference in its entirety.

### FIELD OF THE INVENTION

The present invention relates to the treatment of sleep disordered breathing, and, more particularly, to devices and methods for intra-oral stimulation in the treatment of snoring, sleep apnea, bruxing and temporomandibular joint disorders.

### BACKGROUND OF THE INVENTION

Snoring and Obstructive Sleep Apnea (OSA) are a relatively common sleep disorders that affect from 15 million to as many as 70 million people just in the United States. A patient suffering from OSA literally stops breathing while sleeping possibly for a period of one minute or longer with many patients having hundreds of apneic episodes during the night.

The exact cause of OSA is unclear although when a patient's airway blockage occurs, there is a drop in blood oxygen level with an increase in blood carbon dioxide. As the blood oxygen level decreases, the heart will beat faster trying to compensate for the decrease in blood oxygen to body tissues. Snoring has been reported in the literature to precede OSA. According to a 2006 report from the Institute of Medicine, sleep disorders and sleep deprivation represent a major unmet public health problem in America, with 50 to 70 million people chronically suffering from a disorder of sleep that results in a wide range of deleterious health consequences, including increased risk of hypertension, diabetes, obesity, depression, heart attack, and stroke. Almost 20% of all serious car crash injuries in the general population are associated with driver sleepiness, independent of alcohol effects. It has been reported that the 90% of sleep problem patients are yet undiagnosed.

Current treatments for snoring and OSA include behavioral changes such as losing weight, avoiding alcohol, tobacco, sleeping pills, and attempting to adjust sleeping position. Continuous Positive Airway Pressure (CPAP) can be effective but very uncomfortable and noisy to wear during the night with only 50% patient compliance. Oral appliance therapy is available but many times can cause facial pain, TMD symptoms, and changes in tooth position and occlusion. Surgical approaches are available but most are quite drastic requiring patients to undergo unwanted procedures.

Bruxism is a serious dental problem that involves grinding, gnashing, or clenching of teeth affecting 50%-90% of people. In most adults, stress is a major contributing factor along with anger, frustration, and competition that occur in everyday life. Long term bruxism results in irreversible damage to teeth, both in appearance and function with increasing sensitivity to temperature, possible alveolar bone loss, and eventual loss of teeth. The status of current treatment includes a nightguard to wear while sleeping to protect the teeth from bruxing, but the bruxing continues refocusing destruction on the nightguard. The preferred embodiment of the present invention will mitigate the action of bruxing with electronic stimulation at a subconscious level and not disrupt sleep.

TMD (Temporomandibular Disfunction) is a condition including pain, tenderness, and mal-function of one or both temporomandibular joints (TMJ). This condition reportedly affects 5%-15% of people. Symptoms include; pain in jaw, ear, and or face, clicking, popping, and or locking of the jaw, headache, and uncomfortable or uneven bite. Barring treatment, patients get progressively worse causing irreversible damage to the joint parts. Surgical treatment results have been controversial due to significant risks and unpredictable results. Early non-invasive treatment to prevent irreversible damage to the TMJ with electronic balancing of muscle activity will be provided with this invention.

An example of one approach is presented in U.S. Pat. No. 5,792,067 to Karell which is directed to a device and method for addressing sleep and other disorders through electromuscular stimulation within specific areas of a patient's mouth. A mouthpiece includes an electrode for stimulating either the hard palate, soft palate or the pharynx. The mouthpiece includes a denture-like plate to which the control unit and electrodes may be attached.

### SUMMARY OF THE INVENTION

In view of the foregoing background, it is therefore an object of the present invention to provide effective treatment for snoring and OSA in a patient via an electronic continuous or periodic airway therapy device (ECAT).

This and other objects, features, and advantages in accordance with the present invention are provided by an intra-oral electronic therapy device including a substrate to be positioned in a patient's mouth, a rechargeable battery carried by the substrate, and at least one hamular notch tissue contact electrode extending outwardly from the substrate to contact at least one hamular notch in the patient's mouth. A controller is carried by the substrate and cooperates with the rechargeable battery and the hamular notch tissue contact electrode to provide a predetermined electrical stimulation pattern to a hamular notch in the patient's mouth. A programming interface is carried by the substrate and coupled to the controller to permit programming of the predetermined stimulation pattern therein.

The hamular notch tissue contact electrode may comprise a pair thereof extending rearwardly from the substrate. Also, the predetermined electrical stimulation pattern may be a biphasic electrical stimulation pattern and may include a series of pulses with successive pulses progressively changing in intensity. The programming interface may also be configured to provide recharging of the rechargeable battery.

The substrate may include first and second protective layers, e.g. thermoplastic layers, sealing therebetween the rechargeable battery, controller, and programming interface. An adhesive layer is preferably between the first and second protective layers. The substrate may be adapted to fit within an upper portion of the patient's mouth.

The programming interface may be a wired programming interface, such as an electrical connector exposed on the substrate. The programming interface may be a wireless programming interface such as an inductive coupler, a capacitive coupler, an optical coupler and/or a wireless transceiver.

The controller may further comprises a voltage booster and waveform generator coupled thereto to generate the predetermined electrical stimulation pattern. The controller may also include a battery manager configured to monitor battery conditions.

A method aspect is directed to providing intra-oral electronic therapy and includes providing a substrate to be positioned in a patient's mouth, positioning a rechargeable battery on the substrate, and extending at least one hamular notch tissue contact electrode outwardly from the substrate to contact at least one hamular notch in the patient's mouth. A controller is provided on the substrate and cooperates with the rechargeable battery and the hamular notch tissue contact electrode to provide a predetermined electrical stimulation pattern to the hamular notch in the patient's mouth. The method includes positioning a programming interface on the substrate and coupled to the controller to permit programming of the predetermined stimulation pattern therein.

Thus, effective treatment is provided for snoring and OSA in a patient by opening the airway via flexing or restoring normal muscle tone to the soft palate (levator veli palatini and tensor veli palatini) along with the uvula, tongue, and throat. This action is the result of the delivery of a mild current to the hamular notch by an electronic continuous or periodic airway therapy device (ECAT) for sleep breathing disorders.

### BRIEF DESCRIPTION OF THE DRAWINGS

FIG. 1 is a schematic diagram illustrating a maxillary stone cast with a thin plastic sheet adapted to it and used to fabricate the maxillary plastic arch form for the electronic components of the intra-oral appliance in accordance with the present invention.

FIG. 2 is a schematic diagram illustrating the rechargeable battery and controller located in the palatal aspect of the intra-oral appliance, and the circuit extension leads and contacts which stimulate the hamular notches in accordance with features of the present invention.

FIG. 3A is a bottom view of the intra-oral appliance of FIG. 2 including the electronics being sandwiched between thin protective layers.

FIG. 3B is a cross-sectional view of the intra-oral appliance taken along the line B-B of FIG. 3A.

FIG. 4 is a schematic block diagram illustrating the components of the intra-oral appliance of FIG. 2.

FIG. 5 is a schematic block diagram illustrating features of the programming unit used in cooperation with the intra-oral appliance of FIG. 2.

FIG. 6 is a front view of the exterior of the programming unit of FIG. 5.

FIG. 7 is a back view of the exterior of the programming unit of FIG. 5.

FIG. 8 is a timing diagram illustrating an example of a biphasic square-wave stimulation used in the appliance of FIG. 2.

FIG. 9 is a timing diagram illustrating a rolling intensity stimulation level used in the appliance of FIG. 2.

FIG. 10 is a schematic diagram illustrating another embodiment of the intra-oral appliance for bruxism.

FIG. 11 is a schematic diagram illustrating another embodiment of the intra-oral appliance for TMJ.

FIG. 12 is a schematic diagram illustrating another embodiment of the intra-oral appliance for use on the lower portion of the patient's mouth.

FIG. 13 is a perspective view of a vacuum thermoforming machine used to fabricate the substrate for the appliances of FIGS. 2 and 10-12.

FIG. 14 is a flowchart illustrating various portions of a method of making the appliances of FIGS. 2 and 10-12.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

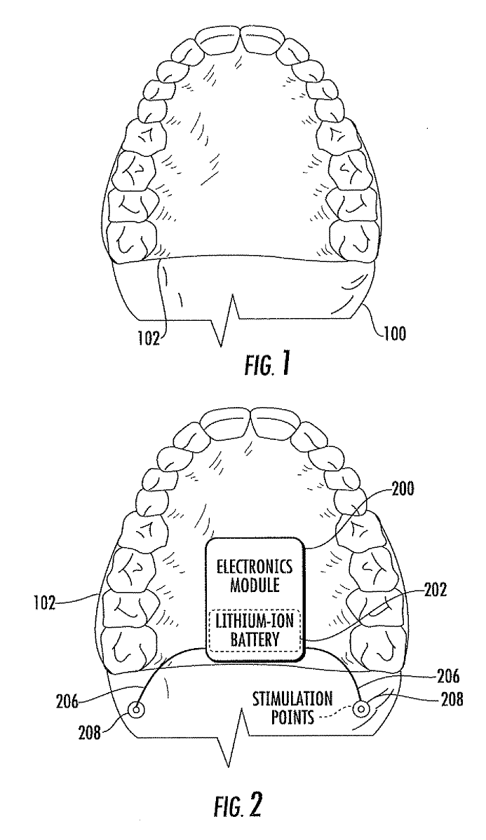

Referring initially to FIG. 1, an illustration of a patient's maxillary teeth is shown. The cast 100 is fabricated by the dentist or dental assistant making impressions (e.g. alginate) of the maxillary and mandibular arches in the usual way impressions are made as would be appreciated by those skilled in the art. A vacuum thermoforming machine (such as manufactured by Raintree Essix Inc., Metairie, La.) can be used to pull down sufficiently heated plastic 102 onto the maxillary model. This plastic material 102 will become the first protective layer upon which components will be mounted. After these components are mounted in the palatal aspect of the arch, a second “sandwiching” piece of thin plastic will be vacuum formed over the electronic components to protect them from saliva as will be described in further detail below.

Referring to FIG. 2, an electronics module 200 is positioned on the formed plastic material 102, and includes a rechargeable battery 202, and circuit extension leads 206 and associated tissues contacts 208 which contact the hamular notches bilaterally in the patient's mouth. The battery 202 is preferably of a sufficient voltage to create the necessary tone in the musculature involved with soft palate flexing or stiffening (tensor veli palatini muscles and the levator veli palatini muscles). Wire leads 206 from the electronics module 200 are preferably 28 gauge wire and run between the “sandwiched” plastic arch form distal to the maxillary 2nd molars and terminate with the circuit extension contacts 208, such as stainless orthodontic ballclamps (0.28 in (0.7 mm)) which contact in the hamular notch.

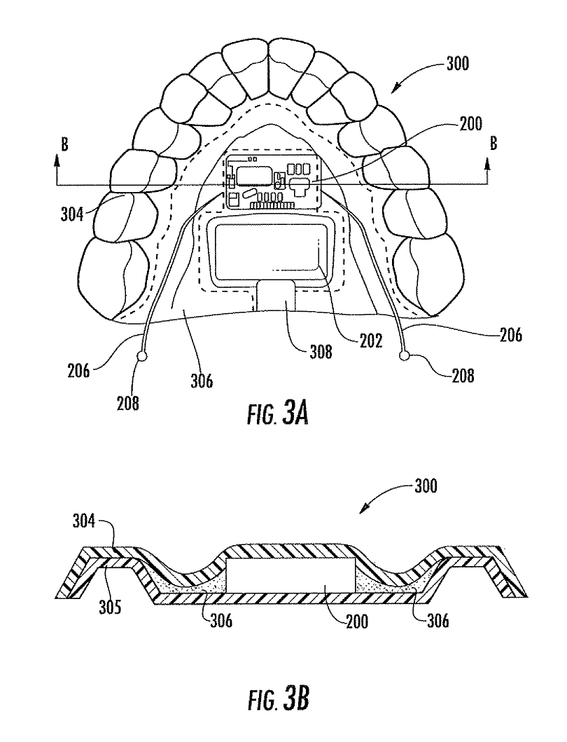

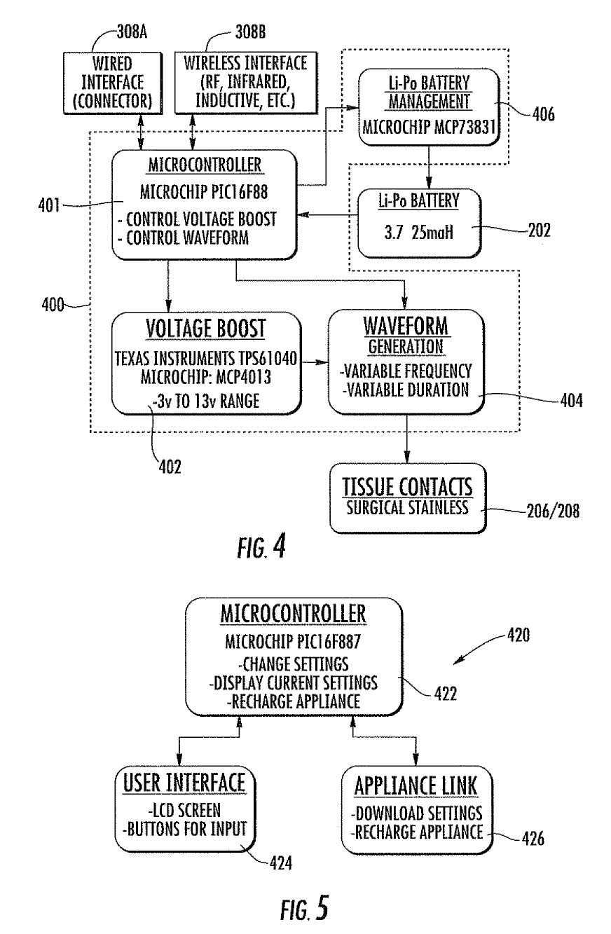

An example of the intra-oral appliance or mouthpiece 300 is illustrated in FIGS. 3A and 3B. The electronics module 200 and battery 202 is sandwiched between upper and lower protective layers 304, 305 (e.g. such as thermoformed plastic layers) for protection of the circuitry from saliva and associated corrosion. Also, an adhesive layer 306 (e.g. a bonded, light-cured, acrylic gel, such as Triad Gel from the Dentsply International of York, Pa.) is preferably applied between the protective layers, e.g. at a periphery thereof, to further aid in the corrosion prevention. An interface 308, such as an electrical connector, is also sealed between the layers 304, 305 and exposed at a periphery thereof.

Accordingly, the appliance 300 defines an intra-oral electronic therapy device including a substrate 304/305 to be positioned in a patient's mouth, a rechargeable battery 202 carried by the substrate, and one or more hamular notch tissue contact electrodes 206/208 extending outwardly from the substrate to contact at least one hamular notch in the patient's mouth. A controller 400 (e.g. referring to FIG. 4) is defined by the electronics module 200 and is carried by the substrate 304/305 and cooperates with the rechargeable battery 202 and the hamular notch tissue contact electrodes 206/208 to provide a predetermined electrical stimulation pattern to a hamular notch in the patient's mouth. A microcontroller 401 and associated programming interface 308 is carried by the substrate 304/305 to permit programming of the predetermined stimulation pattern therein.

The controller 400 may further comprise a voltage booster 402 and waveform generator 404 coupled thereto to generate the predetermined electrical stimulation pattern. The controller may also include a battery manager 406 configured to monitor battery conditions. Illustratively, a lithium-ion battery management IC monitors the battery conditions during charging and use. The charging cycle may be accurately controlled in a constant current mode followed by a constant voltage mode until the battery has been fully recharged. The battery may also be protected against over-voltage, over-current, and under-voltage situations.

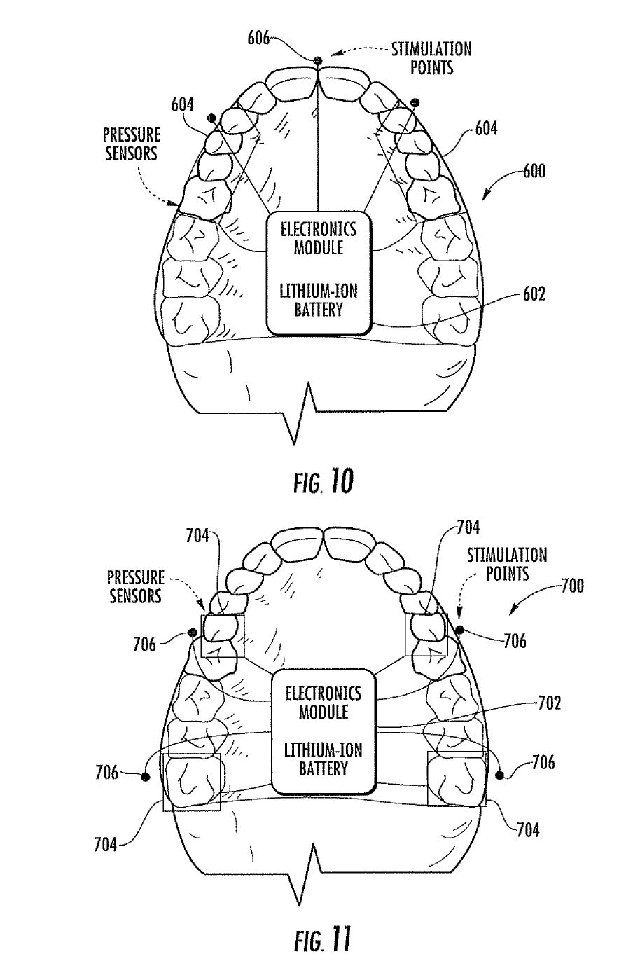

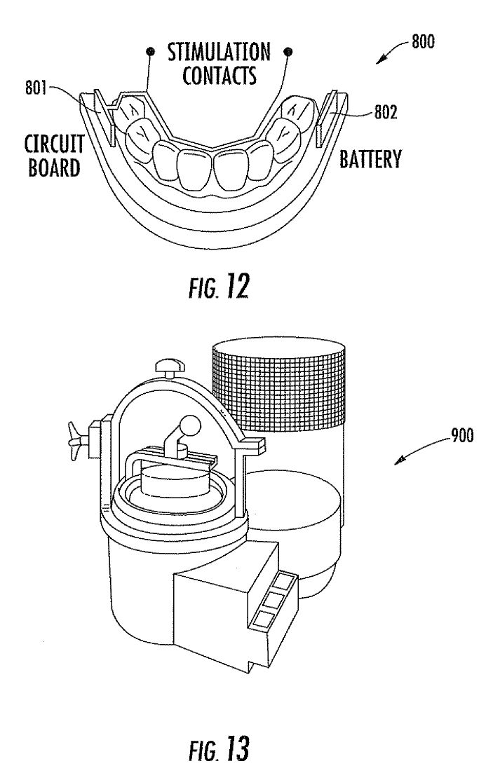

The predetermined electrical stimulation pattern may be a biphasic electrical stimulation pattern and may include a series of pulses with successive pulses progressively changing in intensity as will be described with reference to FIGS. 8 and 9. A low voltage electrical stimulation may be provided by the waveform generator, e.g. via dual push-pull output stages which allow for the creation of a biphasic waveform 500. The biphasic waveform 500 includes alternating, symmetrical positive and negative pulses. Using this type of balanced stimulation may decrease the chance for electrode deterioration and tissue damage.

The waveform generator, e.g. dual push-pull output stages, are supplied with the stimulation voltage level from the voltage boost stage 402. Depending on the mouthpiece settings, a voltage greater than the battery voltage may be required. This may be accomplished with a switching-mode power supply using a boost converter topology. The output of the voltage boost stage may range from 3.5-12.5 volts.

The control of the waveform generator stage 404 and voltage boost stage 402 is managed by the microprocessor 401. This allows for programming of any wave shape with positive and negative components to be generated. The waveform may be bounded by +/− the maximum voltage boost and operating frequency of the microprocessor. An effective waveform has been shown to be a biphasic square-wave 500 at a frequency of 1 kilohertz and 50% duty cycle. The shape, frequency, and duty cycle may all be adjustable.

The stimulation may be applied at periodic intervals ranging from 1-60 seconds. Each stimulation event may have a duration ranging from 100-1000 milliseconds. The microprocessor 401 handles timing of all events based on the settings programmed.

With reference to FIG. 9, the stimulation may use a rolling intensity. This means that each stimulation event is at a different level of intensity. The level of intensity increases or decreases after each stimulation event, staying within the bounds programmed into the microprocessor 401. This has shown to be an effective stimulation event pattern, but any pattern of intensity levels can be programmed into the microprocessor 401.

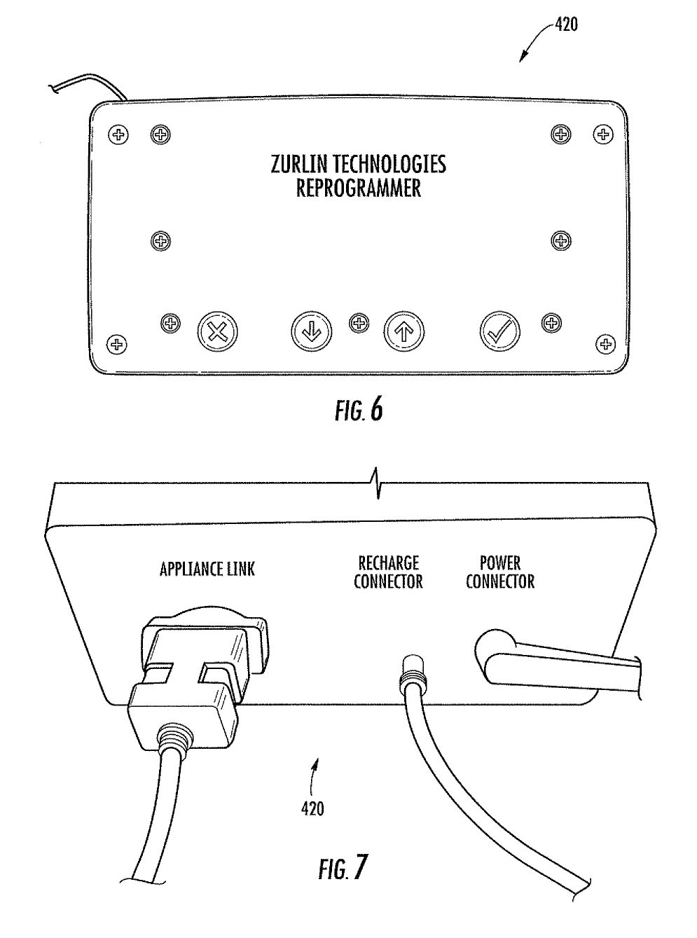

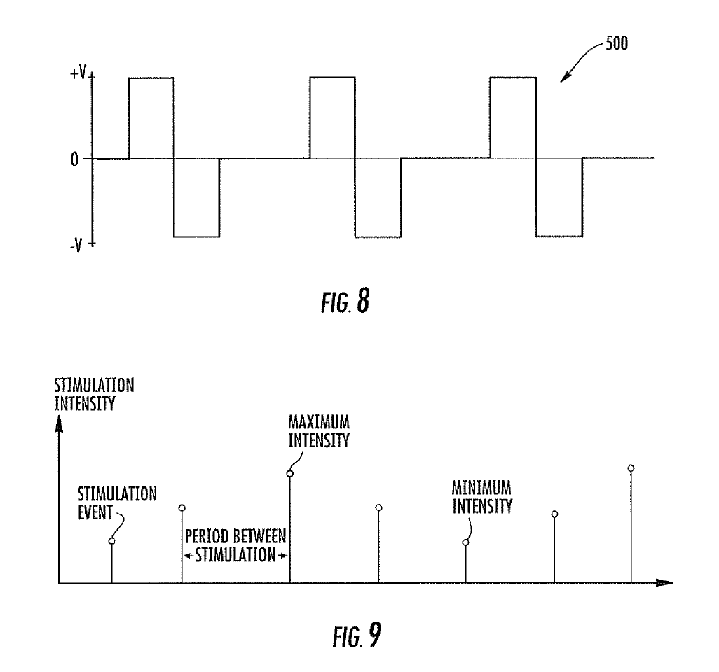

Features of a re-programming unit 420 will be described with reference to FIGS. 5-7. The re-programming unit 420 may include a microcontroller 422 and associated appliance link 426. The re-programming unit 422 allows the user to re-configure settings of the electrical stimulation for the intra-oral appliance 300. The re-programming unit 420 may include user interface 424, e.g. an LCD screen for displaying information, buttons for user input, and various connectors for re-charging and re-programming the appliance. The re-programming unit 420 may also allow the appliance to display its current settings.

The battery 202 may be charged by a physical connection or also by inductive or capacitive charging. The inductive charging requires a pair of coils and capacitors that are tuned to a resonant frequency. A base station coil, e.g. at the re-programming unit 420, is supplied with a signal at its resonant frequency. The coil within the mouthpiece is also tuned to resonate at the same frequency and will receive the signal from the base station coil. The received signal may be rectified to DC and then regulated to 5 volts for the battery charger circuitry.

The intra-oral appliance 300 settings can be transmitted by a direct physical connection, infrared communications or other wireless methods. Communication over the inductive charging coils can be accomplished by using the charging signal as a carrier and modulating data onto that signal. The signal can then be demodulated in the intra-oral appliance 300 to receive the data. As such, the programming interface 308 may also be configured to provide recharging of the rechargeable battery.

A data recorder may be provided in the re=programming unit 420 to monitor snoring/gasping frequency throughout the night. The battery charger feature of the re-programming unit 420 and the associated battery 202 of the intra-oral appliance 300 may utilize connectors manufactured such as 0.100″ pin strip headers and 0.100″ board mount sockets. The socket may be used in the appliance 300 and sealed within the protective thin plastic layers by applying bonded, light-cured, acrylic gel, such as Triad Gel from the Dentsply International of York, Pa., to prevent moisture from entering the mouthpiece. As discussed above, contactless charging, such as electromagnetic, capacitive and/or inductive charging may also be provided instead of the connectors.

Thus, as described, the substrate may be defined by the first and second protective layers 304/305, e.g. thermoplastic layers, sealing therebetween the rechargeable battery 202, controller 400, and programming interface 308. The adhesive layer 306 is between the first and second protective layers. The substrate 304/306 may be adapted to fit within an upper portion of the patient's mouth. Furthermore, the programming interface 308 may be a wired programming interface 308A, such as an electrical connector exposed on the substrate 304/305 (FIG. 4). The programming interface 308 may be a wireless programming interface 308B such as an inductive coupler, a capacitive coupler, an optical or infrared coupler and/or an RF wireless transceiver.

Another aspect of the present invention is directed to the treatment of bruxism. FIG. 10 is an illustration of the intra-oral appliance 600 for treatment of bruxism. This electronic orthosis works as a gnathologic appliance to protect teeth from damage during excursive movements. In addition, the electronics package 602 detects bruxing activity using a pressure electro-conductive rubber sensor or pressure receptor switch 604 such as made by Bridgestone in Tokyo, Japan and stops it with electronic stimulation, via tissue contact 606, to the intra-oral mucosa at a subconscious level without sleep interruption. Patient adjustability is available with the reprogramming unit 420, discussed above, that may be connected, e.g. via wired or wireless communication link, with the intra-oral appliance 600.

Another aspect of the present invention is directed to the treatment of TMJ or TMD. FIG. 11 is an illustration of the intra-oral appliance 700 for treatment of TMJ or TMD. Temporomandibular disorder (TMD), or TMJ syndrome, is a term covering acute or chronic inflammation of the temporomandibular joint, which connects the lower jaw to the skull. This orthotic type appliance 700 detects oral para-functional activity through the use of pressure sensors 704 and an electronics package 702 in the appliance. A pare-functional habit or parafunctional habit is the habitual exercise of a body part in a way that is other than the most common use of that body part. The term is most commonly used by dentists, orthodontists, or maxillofacial specialists to refer to parafunctional uses of the mouth, tongue and jaw. Oral para-functional habits may include bruxism (tooth-clenching or grinding), tongue tension, mouth-breathing, and any other habitual use of the mouth unrelated to eating, drinking, or speaking. Treatment includes electronic stimulation, via tissue contact 706 in response to detected pressure.

Another aspect of the present invention is directed to an intra-oral appliance 800 (FIG. 12) for use in the lower portion of the patient's mouth. The appliance 800 is again fabricated using a bi-laminate plastic sandwich technique but designed to fit on the lower teeth instead of the upper (maxillary) teeth. All electronic components, e.g. electronic circuit 801 and battery 802, are sandwiched between the plastic layers and located on the lateral aspect of the appliance 800. The electronic function is the same as described above, except that the stimulation points may be the tissues in the floor of the mouth near the retro-mylohoid area and under the tongue. The electronic stimulation may restore muscle tone in the tongue, genioglossus, geniohyoid, and palato-pharyngeal muscles to maintain the airway. This lower ECAT appliance 800 can be used in place of the upper ECAT appliance for patients that have an exaggerated gag reflex, very narrow palate, or just cannot tolerate coverage of the roof of the mouth.

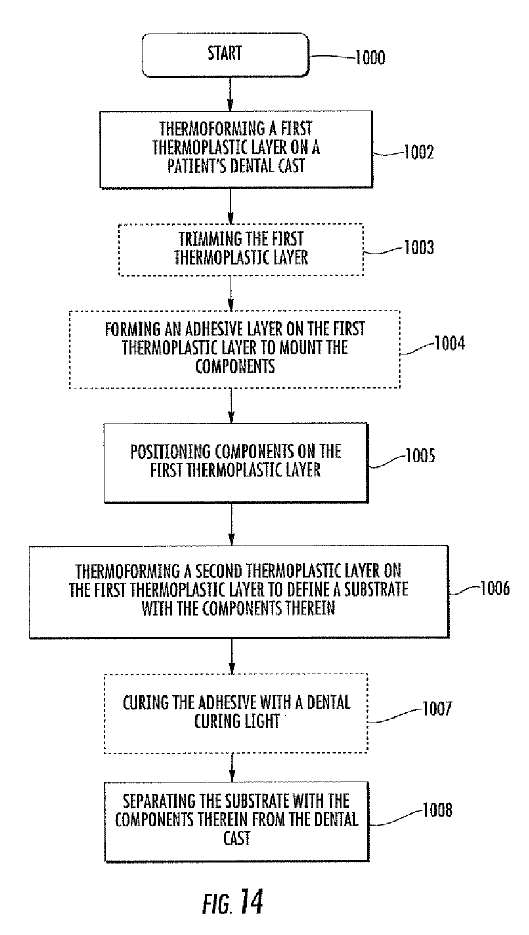

A method aspect will be described with reference to the flowchart in FIG. 14. The method is for making an intra-oral electronic therapy device, e.g. such as illustrated and described with reference to FIGS. 2 and 10-12. The method begins at block 1000 and includes thermoforming a first thermoplastic layer on a patient's dental cast (block 1002), e.g. as received from a patient's dentist, positioning components on the first thermoplastic layer (block 1004), and thermoforming a second thermoplastic layer (block 1006) on the first thermoplastic layer to define a substrate with the components therein. The components include a rechargeable battery 202 (e.g. FIG. 2), at least one tissue contact electrode 206/208 extending outwardly from the substrate to contact at least one tissue area in the patient's mouth, and a controller 400 to cooperate with the rechargeable battery and the at least one tissue contact electrode to provide an electrical stimulation to the at least one tissue area in the patient's mouth. The method includes separating the substrate with the components therein from the dental cast, at block 1008.

The first thermoplastic layer may be trimmed prior to positioning the components thereon (block 1003). Positioning the components may further comprise forming an adhesive layer on the first thermoplastic layer to mount the components (block 1005). The adhesive may comprise a light-curable adhesive, and the method may also comprise curing the light-curable adhesive via a dental curing light (block 1007) after thermoforming the second thermoplastic layer on the first thermoplastic layer.

Additional details of exemplary fabrication techniques for the various embodiments will now be described. First, the fabrication details for the ECAT Snoring/Sleep Apnea Appliance (Upper Teeth) may include the following steps. Upon accurate casts of the patient's teeth, a 2 mm thick foil of Erkoloc Pro bilaminate is thermoformed on the upper teeth using an Erkoform 3-D machine, the occlusion is recorded in this layer by gently closing the cast of the lower teeth into the material while it is soft using the Occluform attachment from Erkodent. This first layer is recovered and excess material is removed with contouring of the base layer with twist drill and acrylic burs. This trimmed first layer is repositioned on the cast to verify fit. An electronics package that may include a circuit board, lithium ion battery, tissue contacts, recharging/re-programming contacts, inductive coil, infra-red receptor, and connecting wires are positioned in the palatal area for best fit. 28 gauge Stainless steel wire is custom bent to the palatal contours and positioned for correct soft tissue contact in the hamular notches bilaterally. A #8 round bur is used to “dimple” the hamular notches to allow for slight compression of the tissue in the mouth. A tight loop is formed in the end of the stainless steel wire to fit the “dimple” in the hamular notches.

The electronics package is set aside and the surface of the first layer is cleaned with an alcohol wipe to remove any contaminates. A thin layer of Triad VLC bonding agents is applied to the surface of this layer and light cured. Triad Clear Gel is applied to circuit board prior to positioning it onto the first layer and light cured. The same sequence is used to permanently place the other parts onto the first layer. A 4 mm ball of hot glue is used to hold the tissue contact loop in the hamular notch so that the wire leads can be covered with gel. The upper cast along with the first layer and the attached electronics is replaced in the Erkoform machine. Another alcohol wipe is used to clean the surface again. Triad VLC Bonding is applied to the surface, and a 1 mm thick foil of Erkodur is thermoformed over this. The occlusion is recorded into this second layer while soft, using the Occluform attachment again. A high intensity curing light is applied to the entire appliance immediately. When cool, the appliance is removed, trimmed and shaped anatomically, and polished.

The fabrication details for ECAT Snoring/Sleep Apnea Appliance (Lower Teeth) may include the following steps. Upon accurate casts of the patient's teeth, a 2 mm thick foil of Erkoloc Pro bilaminate is thermoformed on the lower teeth using an Erkoform 3-D machine. The occlusion is recorded in this layer by gently closing the cast of the lower teeth into the material while it is soft using the Occluform attachment from Erkodent. This first layer is recovered and excess material is removed with contouring of the base layer with twist drill and acrylic burs. This trimmed first layer is repositioned on the cast to verify fit.

An electronics package that may include a circuit board, lithium ion battery, tissue contacts, recharging/re-programming contacts, inductive coil, infra-red receptor, and connecting wires are positioned in the posterior buccal or lingual vestibule area for best fit. 28 gauge Stainless steel wire is custom bent to the oral contours and positioned for correct soft tissue contact in the retro-mylohyoid area and temporarily fixed in position with hot glue. The electronics package is set aside and the surface of the first layer is cleaned with an alcohol wipe to remove any contaminates. A thin layer of Triad VLC bonding agents is applied to the surface of this layer and light cured. Triad Clear Gel is applied to circuit board prior to positioning it onto the first layer and light cured. The same sequence is used to permanently place the other parts onto the first layer. The lower cast along with the first layer and the attached electronics is replaced in the Erkoform machine. Another alcohol wipe is used to clean the surface again. Triad VLC Bonding is applied to the surface, and a 1 mm thick foil of Erkodur is thermoformed over this. The occlusion is recorded into this second layer while soft, using the Occluform attachment again. A high intensity curing light is applied to the entire appliance immediately. When cool, the appliance is removed, trimmed and shaped anatomically, and polished.

The fabrication details for the anti-bruxing appliance may include the following steps. Upon accurate casts of the patient's teeth, a 2 mm thick foil of Erkoloc Pro bilaminate is thermoformed on the upper teeth using an Erkoform 3-D machine. The occlusion is recorded in this layer by gently closing the cast of the lower teeth into the material while it is soft using the Occluform attachment from Erkodent. This first layer is recovered and excess material is removed with contouring of the base layer with twist drill and acrylic burs. This trimmed first layer is repositioned on the cast to verify fit.

An electronics package that may include a circuit board, lithium ion battery, tissue contacts, recharging/re-programming contacts, inductive coil, infra-red receptor, and connecting wires are positioned in the palatal area for best fit. Also, two pressure sensing strips are included in the electronics package which is positioned up the lingual surface of the canines. 28 gauge Stainless steel wire is custom bent to the palatal contours and positioned for correct soft tissue contact in the labial vestibule adjacent to the canines bilaterally. A tight loop is formed in the end of the stainless steel wire to act as the tissue contact and held in place temporarily with a little hot glue.

The electronics package is set aside and the surface of the first layer is cleaned with an alcohol wipe to remove any contaminates. A thin layer of Triad VLC bonding agents is applied to the surface of this layer and light cured. Triad Clear Gel is applied to circuit board prior to positioning it onto the first layer and light cured. The same sequence is used to permanently place the other parts onto the first layer. The upper cast along with the first layer and the attached electronics is replaced in the Erkoform machine. Another alcohol wipe is used to clean the surface again. Triad VLC Bonding is applied to the surface, and a 1 mm thick foil of Erkodur is thermoformed over this. The occlusion is recorded into this second layer while soft, using the Occluform attachment again. A high intensity curing light is applied to the entire appliance immediately. When cool, the appliance is removed, trimmed and shaped anatomically, and polished.

The fabrication details for TMD Appliance may include the following steps. Upon accurate casts of the patient's teeth, a 2 mm thick foil of Erkoloc Pro bilaminate is thermoformed on the upper teeth using an Erkoform 3-D machine. The occlusion is recorded in this layer by gently closing the cast of the lower teeth into the material while it is soft using the Occluform attachment from Erkodent. This first layer is recovered and excess material is removed with contouring of the base layer with twist drill and acrylic burs. This trimmed first layer is repositioned on the cast to verify fit.

An electronics package that may include a circuit board, lithium ion battery, tissue contacts, recharging/re-programming contacts, inductive coil, infra-red receptor, and connecting wires are positioned in the palatal area for best fit. Also, two pressure sensing strips are included in the electronics package which are positioned on the occlusal surfaces from premolar to molar bilaterally. 28 gauge Stainless steel wire is custom bent to the palatal contours and positioned for correct soft tissue contact in the labial vestibule adjacent to the molars bilaterally. A tight loop is formed in the end of the stainless steel wire to act as the tissue contact and held in place temporarily with a little hot glue.

The electronics package is set aside and the surface of the first layer is cleaned with an alcohol wipe to remove any contaminates. A thin layer of Triad VLC bonding agents is applied to the surface of this layer and light cured. Triad Clear Gel is applied to circuit board prior to positioning it onto the first layer and light cured. The same sequence is used to permanently place the other parts onto the first layer. The upper cast along with the first layer and the attached electronics is replaced in the Erkoform machine. Another alcohol wipe is used to clean the surface again. Triad VLC Bonding is applied to the surface, and a 1 mm thick foil of Erkodur is thermoformed over this. The occlusion is recorded into this second layer while soft, using the Occluform attachment again. A high intensity curing light is applied to the entire appliance immediately. When cool, the appliance is removed, trimmed and shaped anatomically, and polished.

Thus, devices and methods are disclosed for intra-oral stimulation in the treatment of snoring, sleep apnea, bruxing and temporomandibular joint disorders.

This application is related to copending patent applications entitled, INTRA-ORAL ELECTRONIC THERAPY DEVICES FOR SLEEP-TREATMENT OF SLEEP-BREATHING DISORDERS, BRUXING DISORDERS, AND ASSOCIATED METHODS, attorney docket no. 60433 and METHODS FOR MAKING INTRA-ORAL ELECTRONIC THERAPY DEVICES FOR TREATING SLEEP-BREATHING DISORDERS, BRUXING DISORDERS, AND TMJ DISORDERS, attorney docket no. 60443 which are filed on the same date and by the same assignee and inventors, the disclosures of which are hereby incorporated by reference.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. An intra-oral electronic therapy device comprising:
a substrate to be positioned in a patient's mouth;
a rechargeable battery carried by said substrate;
at least one hamular notch tissue contact electrode extending outwardly from said substrate to contact at least one hamular notch in the patient's mouth;
a controller carried by said substrate and cooperating with said rechargeable battery and said at least one hamular notch tissue contact electrode to provide a predetermined electrical stimulation pattern to the at least one hamular notch in the patient's mouth; and
a programming interface carried by said substrate and coupled to said controller to permit programming of the predetermined stimulation pattern therein.

2. The intra-oral electronic therapy device of claim 1 wherein said at least one hamular notch tissue contact electrode comprises a pair thereof extending rearwardly from said substrate.

3. The intra-oral electronic therapy device of claim 1 wherein the predetermined electrical stimulation pattern comprises a biphasic electrical stimulation pattern.

4. The intra-oral electronic therapy device of claim 1 wherein the predetermined electrical stimulation comprises a series of pulses with successive pulses progressively changing in intensity.

5. The intra-oral electronic therapy device of claim 1 wherein said programming interface is also configured to provide recharging of said rechargeable battery.

6. The intra-oral electronic therapy device of claim 1 wherein said substrate comprises first and second protective layers sealing therebetween said rechargeable battery, controller, and programming interface.

7. The intra-oral electronic therapy device of claim 6 wherein the first and second protective layers comprise first and second thermoplastic layers.

8. The intra-oral electronic therapy device of claim 6, further comprising an adhesive layer between the first and second protective layers.

9. The intra-oral electronic therapy device of claim 1 wherein said programming interface comprises a wired programming interface.

10. The intra-oral electronic therapy device of claim 9 wherein said wired programming interface comprises an electrical connector exposed on said substrate.

11. The intra-oral electronic therapy device of claim 1 wherein said programming interface comprises a wireless programming interface.

12. The intra-oral electronic therapy device of claim 11 wherein said wireless programming interface comprises at least one of an inductive coupler, a capacitive coupler, an optical coupler and a wireless transceiver.

13. The intra-oral electronic therapy device of claim 1, wherein said controller further comprises a voltage booster and waveform generator coupled thereto to generate the predetermined electrical stimulation pattern.

14. The intra-oral electronic therapy device of claim 1, wherein said controller further comprises a battery manager configured to monitor battery conditions.

15. The intra-oral electronic therapy device of claim 1 wherein said substrate is adapted to fit within an upper portion of the patient's mouth.

16. An intra-oral electronic therapy device comprising:
a substrate to be positioned in a upper portion of a patient's mouth;
a rechargeable battery carried by said substrate;
a pair of hamular notch tissue contact electrodes extending outwardly from said substrate to contact a respective hamular notch in the patient's mouth;
a controller carried by said substrate and cooperating with said rechargeable battery and said at least one hamular notch tissue contact electrode to provide a predetermined electrical stimulation pattern to the at least one hamular notch in the patient's mouth;
said substrate comprising first and second thermoplastic layers sealing therebetween said rechargeable battery, controller, and programming interface; and
an adhesive layer between the first and second protective layers.

17. The intra-oral electronic therapy device of claim 16 wherein the predetermined electrical stimulation pattern comprises a biphasic electrical stimulation pattern including a series of pulses with successive pulses progressively changing in intensity.

18. The intra-oral electronic therapy device of claim 16 further comprising an electrical connector exposed on said substrate and defining a wired programming interface carried by said substrate and coupled to said controller to permit programming of the predetermined stimulation pattern therein.

19. The intra-oral electronic therapy device of claim 16, wherein said controller further comprises a voltage booster and waveform generator coupled thereto to generate the predetermined electrical stimulation pattern.

20. The intra-oral electronic therapy device of claim 16, wherein said controller further comprises a battery manager configured to monitor battery conditions.

21. A method of providing intra-oral electronic therapy comprising:
providing a substrate to be positioned in a patient's mouth;
positioning a rechargeable battery on the substrate;
extending at least one hamular notch tissue contact electrode outwardly from the substrate to contact at least one hamular notch in the patient's mouth;
providing a controller on the substrate and cooperating with said rechargeable battery and the at least one hamular notch tissue contact electrode to provide a predetermined electrical stimulation pattern to the at least one hamular notch in the patient's mouth; and
positioning a programming interface on the substrate and coupled to the controller to permit programming of the predetermined stimulation pattern therein.

22. The method of claim 21, wherein the at least one hamular notch tissue contact electrode comprises a pair thereof extending rearwardly from the substrate.

23. The method of claim 21, wherein the predetermined electrical stimulation pattern comprises a biphasic electrical stimulation pattern.

24. The method of claim 21, wherein the predetermined electrical stimulation comprises a series of pulses with successive pulses progressively changing in intensity.

25. The method of claim 21, wherein said programming interface is also configured to provide recharging of said rechargeable battery.

26. The method of claim 21, wherein said substrate comprises first and second protective layers sealing therebetween said rechargeable battery, controller, and programming interface.

27. The method of claim 26, wherein the first and second protective layers comprise first and second thermoplastic layers.

28. The method of claim 26, further comprising an adhesive layer between the first and second protective layers.

29. The method of claim 21, wherein said programming interface comprises an electrical connector exposed on said substrate.

30. The method of claim 21, wherein said programming interface comprises at least one of an inductive coupler, a capacitive coupler, an optical coupler and a wireless transceiver.

31. The method of claim 21, wherein said controller further comprises a voltage booster and waveform generator coupled thereto to generate the predetermined electrical stimulation pattern.

32. The method of claim 21, wherein said controller further comprises a battery manager configured to monitor battery conditions.

33. The method of claim 21, wherein said substrate is adapted to fit within an upper portion of the patient's mouth.
